(19) United States
(12) Patent Application Publication
(10) Pub. No.: US 2024/0286140 A1
(43) Pub. Date: Aug. 29, 2024

(54) DEVICE FOR HOLDING AND LIMITING THE ROTATION OF A BLOOD-COLLECTION TUBE OR OF A CYLINDRICAL CONTAINER FITTED IN A CAVITY

(71) Applicant: Diagnostica Stago, Asnieres Sur Seine (FR)

(72) Inventor: Benoit LAGARDE, Sannois (FR)

(21) Appl. No.: 18/290,134

(22) PCT Filed: May 6, 2022
(86) PCT No.: PCT/EP2022/062363

§ 371 Date: Nov. 9, 2023

(30) Foreign Application Priority Data
May 12, 2021 (FR) FR2105057

(51) Int. Cl. B01L 9/00; A61B 5/15; A61J 1/16

(52) U.S. Cl. CPC B01L 9/52; A61B 5/150343; A61J 1/16; B01L 2200/025; B01L 2300/021

(57) ABSTRACT

An elastic-ring device for holding biological containers in position for the automatic reading of identification codes or of information printed directly on the containers or on labels applied to the containers, and more particularly to automated identification of blood-collection sample tubes and additionally other types of biological-product containers passing through the automated system.

### FIELD OF APPLICATION OF THE INVENTION

The invention relates to a device and a method for holding blood analysis tubes or vials of biological products using a system of elastic rings positioned in different ways, within individual carriers or racks for transporting several containers. These rings keep the containers oriented in the initial position within the carriers over time. These rings are provided in the form of inserts placed in the tube or vial carriers, with different assembly arrangements enabling the holding function to be adapted as required.

This invention applies non-exclusively to the vertical holding of medical analysis tubes and preventing the rotation thereof in carriers, while enabling the insertion and removal of the tubes in the cavities in which said tubes are positioned. One of the uses of this method for holding tubes in position relates to the reading of information written on the labels of the biological sample analysis tubes and vials of liquid or solid products.

The information available is the form of characters printed on medium, read by OCR or using bar codes read by a reader. Other information may also relate to the state of the container, and to measurements of characteristics of the liquid or solid elements in the container, where such measurements require specific positioning.

### CONTEXT

The present invention relates to a method and a device for holding biological containers in position for the automatic reading of identification codes or information printed directly on the containers or on labels affixed to the containers.

The invention relates more particularly to the automated identification of blood sample collection tubes and additionally to other types of containers of biological products transiting on the automated system. The blood samples are mainly packed in test tubes in the form of whole blood or centrifuged blood, with the plasma in the upper part.

These blood samples are intended for analysis and must be strictly associated with a patient record. An identification code is paired with the tube or vial to ensure the traceability and information transfer required for the proper execution of diagnostic operations. This identification code is positioned mainly on a label (or printed on the tube itself) and oriented so as to be fully visible in front of the reading window of the reader. The label is manually positioned on the tube or vial at variable locations on the cylindrical portion of the container.

Tubes or vials provided with labels are placed in single carriers or multi-position racks with windows for access to information.

In an in vitro diagnostic apparatus or in a blood collection tube conveying chain, the angular orientation of the tubes is essential to enabling the patient identification label on the tube to be read. The label contains important information about the biological sample and must be acquired by the automated chain or by the diagnostic instrument used to carry out the technical operations or analyses. This identification of the tube by reading the label is mainly carried out by a bar code reader, or by an OCR character reader positioned opposite the label to be read.

Any rotation of the tube between the initial insertion position and the phase in which the identifier is read by the bar code reader is liable to degrade or prevent the reading of the information.

Accidental rotation of the tube may render the label reading system inoperative, since the label would then be partially truncated or totally hidden and not readable by the reader. These changes in the angular positioning of the tubes may occur, as a non-exhaustive example, as a result of handling of the carriers, exposure to vibrations, involuntary friction, or forces capable of generating rotation about the vertical ascending axis of the tubes or vials.

In a tube conveying chain using single pucks, or conveying racks carrying several tubes, the tubes are subjected to shocks during transport and tend to rotate inside the respective housing, changing the initial orientation of the tubes.

There are several solutions to compensate for these technical limitations on medical systems:
A first particularly efficient method involves reorienting the tube by means of a system that rotates the tube until the bar code reader reads the label moving in front of the reader (patent FR2764704 Diagnostica STAGO Dec. 18, 1998).
A second method completely eliminates this concept in Workcell Max hemostasis cells, which implements double tube reorientation: upon exit from the centrifuge and then upon entry into the instrument.
A third method involves inserting the tube with high mechanical holding forces along the radial axis, using rings or clamps to ensure the centering and clamping of the tube. This method has the advantage of locking the angular position of the tube and thus reducing the need for the tube reorientation system in order to save cycle time or limit the use of the reorientation system. This method has the drawback of creating significant friction on the label, which increases the insertion forces and impairs the ability to insert and remove the tube in the cavity. This solution is poorly suited to tubes of variable diameters, for which the holding and insertion forces are poorly controlled.
A fourth method involves using clamps that lock and unlock the tube holding system during insertion and removal operations. This method is complex and expensive.
A fifth method involves reading the identification tag using a manual reader, by positioning the tag opposite the reader. This method requires the intervention of an operator for each tube to be read, which is unacceptable for automated processing.

In order to eliminate the drawbacks of the current solutions in chains not covered by the first two methods, the proposed invention makes it possible to ensure the total holding of the tube (zero rotation) or the partial holding of the tube (permitting identified movements) without using external actuators and limiting the tube insertion or extraction forces.

The main application of this method is:
Loading sample tubes into the cavities of different types of carriers (racks, carriers, pucks, static station), the identification of the sample tube being associated with the patient concerned on a label including a bar code,
The tube is oriented to optimize the bar code reading conditions,
The tubes are provided in carriers in the form of tube racks (or single pucks) to convey the tubes to the analytical instruments,
Accepting tubes and placement in front of the reading system, without changing the initial tube positioning,
Reading of the code to ensure the traceability of the tube throughout the process in the automated chain or in the automated analyzer,
Carrying out technical or diagnostic operations taking into account the identifier on the tube,
Sample tubes can be unloaded manually or automatically using different systems, depending on the subsequent uses of the tubes. The tube removal forces must be as low as possible.

The systems usually used for holding tubes in cavities have several drawbacks:
The systems usually involve locking along the radial and vertical axes, generating large insertion and extraction forces,
Rotational and translational friction is often caused by the same element,
Variations in tube diameters generate uncontrolled holding forces,
Variations in the coefficient of friction result in variable holding forces,
Label thicknesses result in significant variations in holding forces,
The insertion and extraction forces of the tubes are not particularly reproducible, depending on the diameters of the tubes,
Rotation cannot be permitted in one direction and prevented in the other direction.

The present invention relates to a method for limiting the rotation about the main axis of revolution of a cylindrical container (blood collection tube or vial) when positioned in a cavity provided for this purpose.

The invention comprises a method for locking a tube (T) in one or both directions of rotation and technical means, specifically the insert (I), which is added to the rack (PS or PD), the puck or the carrier.
The invention also enables the inserted tube to be held vertically by a calibrated holding force, inserted and extracted with limited force.
The invention can be implemented specifically in different forms to ensure the desired combinations of tube rotation prevention, in one direction or in both directions.
The invention can be implemented by means of an insert (I), which is added into a cavity (C).
The insert can be of different shapes and sizes to suit the different shapes of carrier used.

According to the method usually used, the collection tube is handled via the upper portion thereof by an operator or a gripping system entering an automatic installation, to be inserted into a cavity designed to receive the tube. The method for treating patient blood sample collection tubes is intended to distribute, position and angularly orient said tubes in different cells, which provide a reference position, making it possible to carry out different operations.

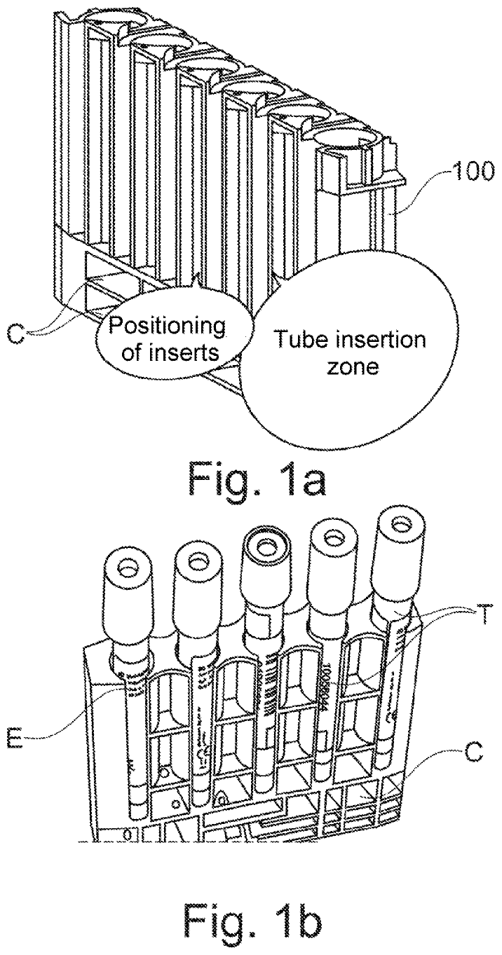

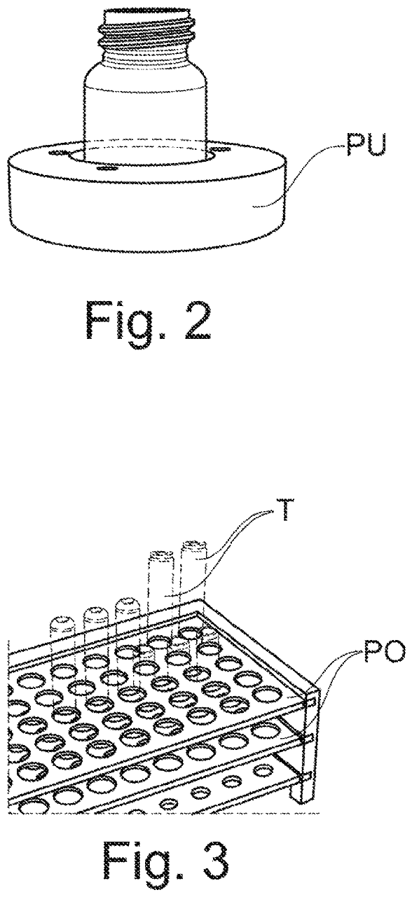

These cells for receiving the collection tubes are made in:
Transport racks for several tubes (T) for automated analysis tube processing (FIGS. 1a and 1b),
Unitary transport systems for tubes or cylindrical containers, referred to as pucks (PU) in the remainder of the document (FIG. 2),
Laboratory carriers (PO) for positioning tubes (T) for operators (FIG. 3),
In static-position carriers in instruments or automated chains intended to receive and store tubes for a certain period of time, pending further operations.

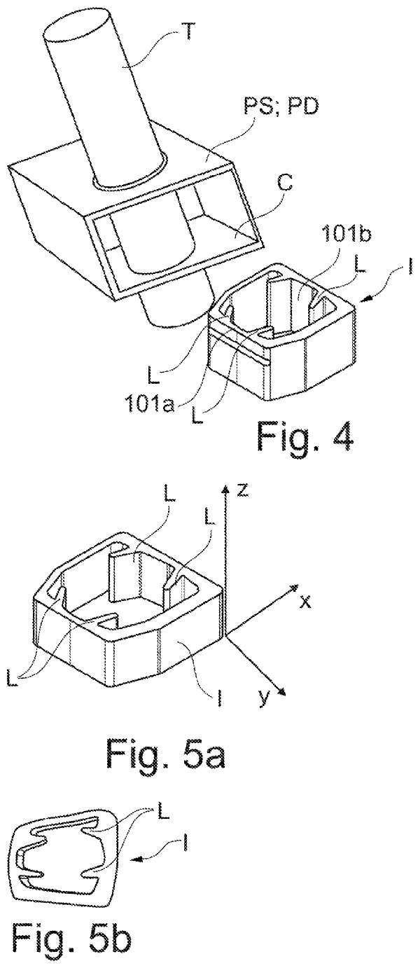

The invention relates to a rotation-prevention insert system for positioning a cylindrical container (a tube or a vial) in the cells, enabling the tube to be inserted in a pre-defined angular position and ensuring that the angular position of the tube does not change over time (FIG. 4).

The insert system (FIGS. 5a and 5b) provides:
A wedging function provided by a flexible one-piece assembly ensuring a friction-based locking function, which remains stable regardless of the intensity of any destabilizing force, provided that this force does not change direction and remains perpendicular to the ascending vertical axis.
Ease of insertion and proper positioning of tubes within the cavities: the friction force of the lips or the wedging force has to be reduced to enable the insertion and extraction of the tubes from the cavities intended to receive the tubes, without degrading the system and without generating significant stripping forces.
The rotation-prevention function of the tubes is achieved by a system of lips (also referred to as blades) wedging against the outer faces of the tube in contact with said lips.

The lips (L) are positioned in a direction that permits rotation in one direction only and prevents rotation in the other direction.

The lips (L) are made of flexible materials and with a high coefficient of friction to enable wedging.

The rigidity/flexibility of the lips (L) enables wedging, without any risk of the lips turning over.

The materials and surface textures are chosen to create the friction required for wedging in one direction and enabling rotation in the other direction.

The lips can vary in number, shape and position.
The function of positioning the rotation-prevention insert (I) in the rack (PS; PD) or in the puck (PU) is achieved by a housing in the lower portion of the rack, which makes it possible to position the insert.

The insert is held in the vertical direction z by one wall or two ribs,

The insert is positioned along the axes x, y by the walls of the housing,

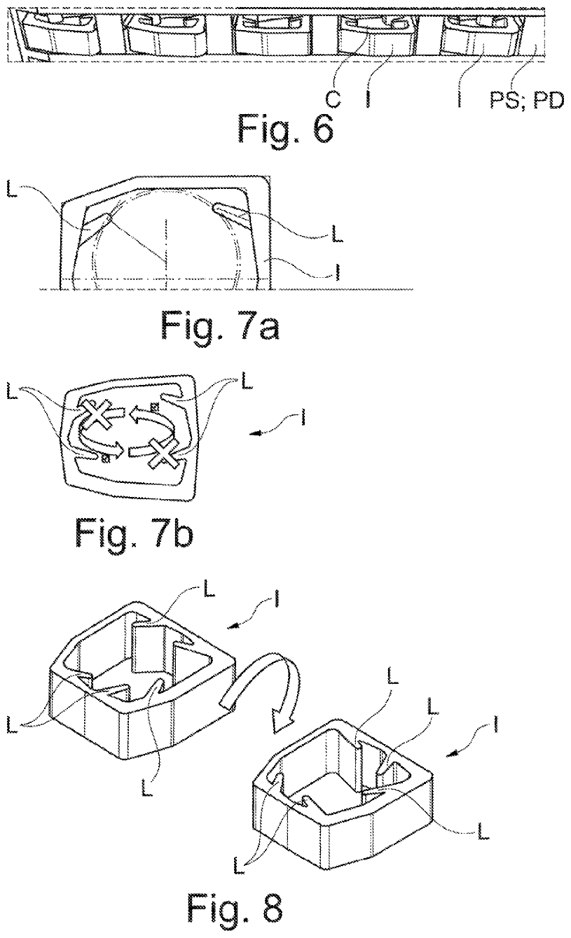

The insert is prevented from rotating by fitting against the walls (FIG. 6),

The housing can take many forms: a cavity with lateral insertion, a housing enabling captive insertion, a location for sealing the insert therein.
A lip shape providing a free-wheel function (FIGS. 7a and 7b).

A rotation-prevention function when approached from the top,

A guidance function when approached from the bottom,
The configuration of the tube locking function is associated with the shape of the insert.

The insert may include lips in a single direction, rotation in the clockwise direction, or locking in the counterclockwise direction, and vice versa when the insert is turned symmetrically about the horizontal plane (FIG. 8).

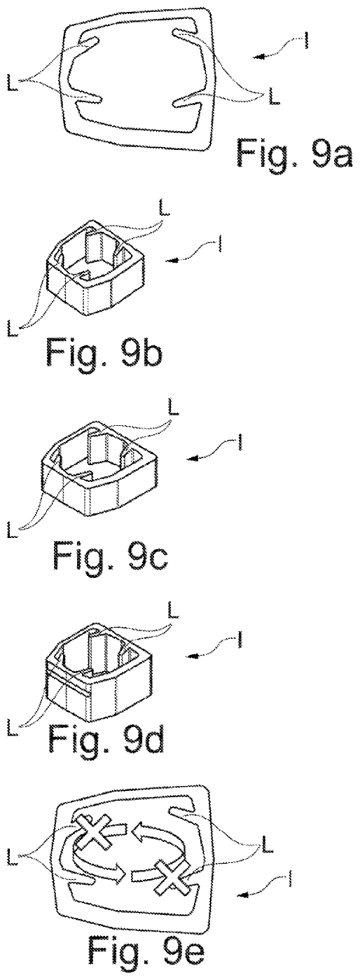

The insert may include lips in both locking directions (clockwise and counterclockwise) preventing all rotation of the cylindrical container (FIGS. 9a to 9e).

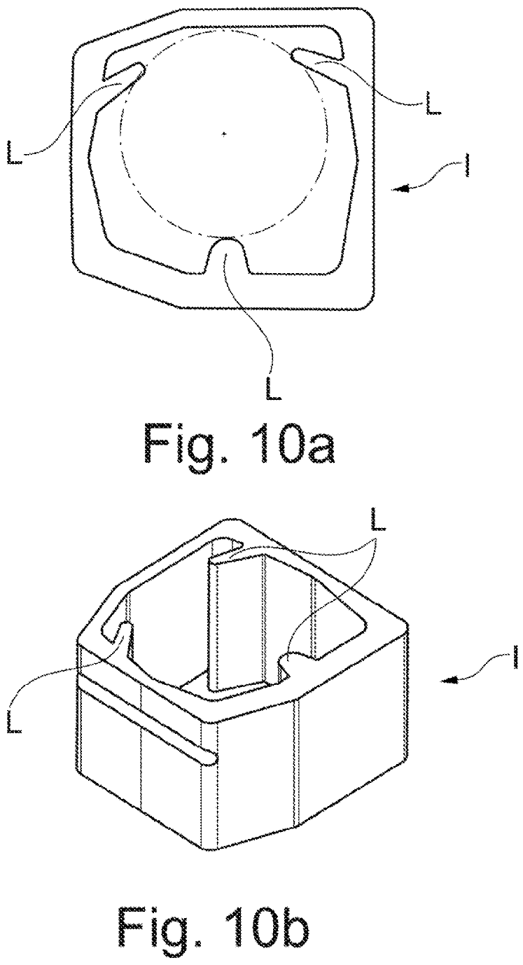

The insert may include a combination of lips and bearing points on the generators providing a geometrical reference of the container to be immobilized. (FIGS. 10a and 10b).

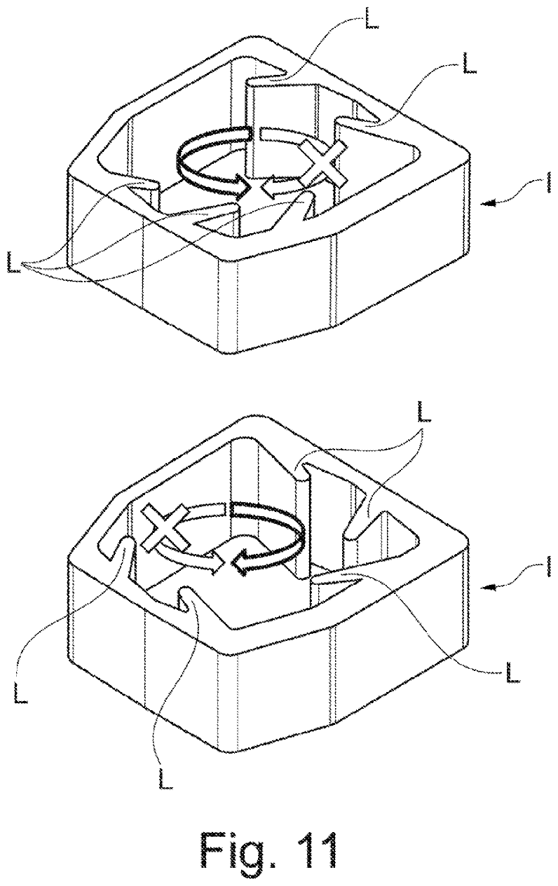

Two inserts preventing rotation in a single direction can be superposed head to tail to prevent rotation in both directions (FIG. 11).
The method using the lip system has eight main functions:
An insert shape designed to be placed in cavities provided for this purpose to prevent the rotation of said insert.
An insert that can include a variable number of lips organized as a function of the required rotation and rotation prevention.
A method for assembling inserts creating a rotation-prevention function that can be configured to suit different rack cavities.
Lip shapes ensuring lip flexion and providing the required wedging.
Lip orientations enabling a stable position to be maintained over time.
A coefficient of friction enabling the wedging function of the lips on the tubes.
Variable insert-height positioning as a function of the technical environment.
A flexible-lip solution providing a single common response to variations in the external tube diameters.

FIGS. 1a and 1b show a tube transport rack 100 defining insertion zones for the tubes T that open upward, in this case for six vertical tubes in the example illustrated. Each tube receiving zone is open on one side to show the label E carried by the tube T. The rack 100 also defines cavities C for receiving inserts, through which the corresponding tubes pass to enable the insert to act on the tube.

FIG. 4 partially shows a support portion (for example a rack PS or PD) for a tube T defining a cavity C and the corresponding insert I intended to be inserted into this cavity C. The insert I shown in FIG. 4 comprises lips L arranged in pairs on two opposing inner faces 101a and 101b. The lips L of each pair are oriented away from each other toward the inside of the insert. These lips L are applied to the tube T passing through the insert, and each lip extends over the entire height of the insert in contact with the tube T.

Depending on the shape and orientation of the lips, the rotation of the tube can be opposed to a greater or lesser extent at the contact of the lips about the axis of the tube. The blades oriented in the direction of rotation of the tube tend to apply a lower friction force than the blades oriented in the opposite direction, since the latter tend to wedge against the tube during this opposite rotation. In other words, the friction force between the tube and the blade, where the blade is oriented opposite to the direction of rotation of the tube, tends to deform the blade such as to further increase the pressure exerted by the blade on the tube and therefore the friction of the blade on the tube. Where the blade is oriented in the direction of rotation, the blade cannot be wedged in this way.
The insert may be made of a thermoplastic material.
Features of different aspects of the invention are given below as points. Each point relates to separate subject matter of the invention, which may be combined with other points or features of the invention disclosed above.

Point 1. A device for positioning and preventing the rotation of cylindrical receptacles (T) in cells of carriers in a static position (PS) or in motion (PD),
characterized in that it uses one or more inserts (I) for centering and holding in position the cylindrical receptacles (T), using the principle of wedging deformable blades (L) to prevent the rotation of the cylindrical receptacles (T), to ensure the correct positioning of the cylindrical receptacle when reading information on the walls of the tube or taking measurements inside the tube.

Point 2. Device according to point 1,
characterized in that the insert (I) includes deformable blades (L) oriented in a preferential direction such as to provide a wedging function to prevent rotation in one direction and a guidance function in the opposite direction.

Point 3. Device according to point 1,
characterized in that the insert (I) fits into a cavity (C) provided for this purpose in the carrier (PS or PD).

Point 4. Device according to point 2,
characterized in that the shape and coefficient of friction of the deformable blades (L) provide a wedging function in one direction of rotation (clockwise or counterclockwise) and a free movement function in the opposite direction of rotation (respectively counterclockwise or clockwise).

Point 5. Device according to point 2,
characterized in that the shape and rigidity of the deformable blades (L) prevent said deformable blades from folding back onto themselves during wedging.

Point 6. Device according to point 2,
characterized in that the shape and finish of the deformable blades (L) encourage the vertical sliding of the cylindrical receptacle (T) in order to limit the related insertion and stripping forces.

Point 7. Device according to point 2,
characterized in that there are typically four deformable blades (L), but there may be any even or odd number of deformable blades dimensioned as a function of the rotation-prevention forces sought.

Point 8. Device according to point 2,
characterized in that the deformable blades (L) are all oriented in the same direction to allow rotation in one direction and to prevent rotation in the other direction.

Point 9. Device according to point 2,
characterized in that the deformable blades (L) are oriented in opposite directions in order to completely prevent rotation in both clockwise and counterclockwise directions.

Point 10. Device according to point 2,
characterized in that some deformable blades (L) are replaced by bearing points to bear the load and ensure precise positioning.

Point 11. Device according to point 2,
characterized in that the insert (I) is totally symmetrical with respect to the horizontal plane to provide a reversible locking function with a single insert shape.

Point 12. Device according to point 2,
characterized in that the insert (I) is entirely free to rotate in the cavity (C) and thus only performs a centering function for tubes of variable diameters and free rotation.

Point 13. Device according to point 3,
characterized in that the inserts (I) are removable from the cavity (C) provided for this purpose.

Point 14. Device according to point 3,
characterized in that the cavity (C) provided for this purpose in the carrier (PS or PD) makes it possible to accommodate one or two inserts.

Point 15. Device according to point 3,
characterized in that the vertical travel of the insert (I) is limited by ribs or by bearing planes in the cavity (C) provided for this purpose in the carrier (PS or PD).

Point 16. Device according to point 3,
characterized in that the rotation of the insert (I) is limited by walls against which the insert (I) bears.

Point 17. Device according to point 3,
characterized in that the cavity (C) is a recess or is shaped to hold the insert along the different axes of the Cartesian reference system.

Point 18. Device according to point 3,
characterized in that the cavity (C) provided for this purpose in the carrier (PS or PD) makes it possible to accommodate one or two inserts, and can be divided in the middle by a rib.

Point 19. Device according to point 3,
characterized in that the inserts (I), where said inserts have a symmetrical shape, may be installed opposite each other in the cavity (C) to prevent rotation in both directions.

Point 20. Device according to point 2,
characterized in that the inserts (I) have a natural demolding shape where produced by plastic injection molding or extrusion and cutting.

Point 21. An insert and tube assembly comprising:
a support frame,
several elastically deformable blades (L) carried by the frame and arranged to bear against and clamp the tube when said tube is engaged through the insert.

Point 22. The assembly according to point 21, in which all of the blades (L) are oriented in the same circumferential direction about the axis of the tube, so as to oppose the rotation of the tube with a frictional torque that is greater in one direction than in the opposite direction.

Point 23. The assembly according to point 21, in which the blades (L) are oriented in opposite circumferential directions so as to oppose rotation of the tube in each direction of rotation by wedging.

Point 24. The assembly according to any one of points 21 to 23, in which the blades (L) extend over the entire height of the insert (I).

Point 25. The assembly according to any one of points 21 to 24, in which the blades (L) have rounded edges in contact with the tube.

Point 26. The assembly according to any one of points 21 to 25, in which the thickness of the blades (L) increases toward the support frame.

Point 27. The assembly according to any one of points 21 to 26, in which the support frame has two outer faces converging toward one side of the frame.

Point 28. The assembly according to point 21, comprising two first inclined blades converging toward a first side of the frame and connecting to respective opposing sides thereof, and a third blade connecting to a side of the frame opposite the first side, the circumference of the tube bearing against the first two blades and against the third blade.

Point 29. The assembly according to point 21, comprising five blades oriented in the same circumferential direction, two pairs of blades arranged on respective opposing sides and one blade on one side connecting these two sides.

Point 30. A carrier having an insert (I) and at least one cavity (C) for receiving the insert, the insert comprising:
a support frame, and
several deformable blades (L) carried by the frame and arranged to bear against and clamp the tube when said tube is engaged through the insert.

Point 31. The carrier according to point 30, comprising at least one passage for the tube (T) above the cavity (C).

Point 32. The carrier according to one of points 30 and 31, comprising a plurality of cavities (C) arranged side by side, notably at least five cavities.

Point 33. A method for placing at least one tube (T) in a tube transport rack (100) defining receiving zones for tubes (T) that open upward, the rack (100) comprising a plurality of carriers according to any one of claims 30 to 32, in which said tube (T) is inserted in a receiving zone in such a way that the corresponding insert (I) can act on the tube (T) and prevent the rotation of said tube about the longitudinal axis thereof in one or both directions of rotation.

The device according to the invention can be applied to product racks or to product pucks, in order to hold vials of biological products in position. This application may be advantageous for stirring directly in a rack (reciprocating motion, for example) or indirectly within a standalone puck set in motion.

1. A device for positioning and preventing the rotation of cylindrical receptacles in cells of carriers in a static position or in motion, comprising one or more inserts for centering and holding in position the cylindrical receptacles, using the principle of wedging deformable blades to prevent the rotation of the cylindrical receptacles, to ensure the correct positioning of the cylindrical receptacle when reading information on the walls of the tube or taking measurements inside the tube.

2. The device as claimed in claim 1,
wherein the insert includes deformable blades oriented in a preferential direction such as to provide a wedging function to prevent rotation in one direction and a guidance function in the opposite direction.

3. The device as claimed in claim 1,
wherein the insert fits into a cavity provided for this purpose in the carrier.

4. The device as claimed in claim 2,
wherein the shape and coefficient of friction of the deformable blades provide a wedging function in one direction of rotation and a free movement function in the opposite direction of rotation.

5. The device as claimed in claim 2,
wherein the shape and rigidity of the deformable blades prevent said deformable blades from folding back onto themselves during wedging.

6. The device as claimed in claim 2,
wherein the shape and finish of the deformable blades encourage the vertical sliding of the cylindrical receptacle in order to limit the related insertion and stripping forces.

7. The device as claimed in claim 2,
wherein there are typically four deformable blades, but there may be any even or odd number of deformable blades dimensioned as a function of the rotation-prevention forces sought.

8. The device as claimed in claim 2,
wherein the deformable blades are all oriented in the same direction to allow rotation in one direction and to prevent rotation in the other direction.

9. The device as claimed in claim 2,
wherein the deformable blades are oriented in opposite directions in order to completely prevent rotation in both clockwise and counterclockwise directions.

10. The device as claimed in claim 2,
wherein some deformable blades are replaced by bearing points to bear the load and ensure precise positioning.

11. The device as claimed in claim 2,
wherein the insert is totally symmetrical with respect to the horizontal plane to provide a reversible locking function with a single insert shape.

12. The device as claimed in claim 2,
wherein the insert is entirely free to rotate in the cavity and thus only performs a centering function for tubes of variable diameters and free rotation.

13. The device as claimed in claim 3,
wherein the inserts are removable from the cavity provided for this purpose.

14. The device as claimed in claim 3,
wherein the cavity provided for this purpose in the carrier makes it possible to accommodate one or two inserts.

15. The device as claimed in claim 3,
wherein the vertical travel of the insert is limited by ribs or by bearing planes in the cavity provided for this purpose in the carrier.

16. The device as claimed in claim 3,
wherein the rotation of the insert is limited by walls against which the insert bears.

17. The device as claimed in claim 3,
wherein the cavity is a recess or is shaped to hold the insert along the different axes of the Cartesian reference system.

18. The device as claimed in claim 3,
wherein the cavity provided for this purpose in the carrier makes it possible to accommodate one or two inserts and can be divided in the middle by a rib.

19. The device as claimed in claim 3,
wherein the inserts, where said inserts have a symmetrical shape, are installed opposite each other in the cavity to prevent rotation in both directions.

20. The device as claimed in claim 2,
wherein the inserts have a natural demolding shape where produced by plastic injection molding or extrusion and cutting.

21. An insert and tube assembly comprising:
a support frame,
several elastically deformable blades carried by the frame and arranged to bear against and clamp the tube when said tube is engaged through the insert.

22. The assembly as claimed in claim 21, in which all of the blades are oriented in the same circumferential direction about the axis of the tube, so as to oppose the rotation of the tube with a frictional torque that is greater in one direction than in the opposite direction.

23. The assembly as claimed in claim 21, in which the blades are oriented in opposite circumferential directions so as to oppose rotation of the tube in each direction of rotation by wedging.

24. The assembly as claimed in claim 21, in which the blades extend over the entire height of the insert.

25. The assembly as claimed in claim 21, in which the blades have rounded edges in contact with the tube.

26. The assembly as claimed in claim 21, in which the thickness of the blades increases toward the support frame.

27. The assembly as claimed in claim 21, in which the support frame has two outer faces converging toward one side of the frame.

28. The assembly as claimed in claim 21, comprising two first inclined blades converging toward a first side of the frame and connecting to respective opposing sides thereof, and a third blade connecting to a side of the frame opposite the first side, the circumference of the tube bearing against the first two blades and against the third blade.

29. The assembly as claimed in claim 21, comprising five blades oriented in the same circumferential direction, two pairs of blades arranged on respective opposing sides and one blade on one side connecting these two sides.

30. A carrier having an insert and at least one cavity for receiving the insert, the insert comprising:
a support frame, and
several deformable blades carried by the frame and arranged to bear against and clamp the tube when said tube is engaged through the insert.

31. The carrier as claimed in claim 30, comprising at least one passage for the tube above the cavity.

32. The carrier as claimed in claim 30, comprising a plurality of cavities arranged side by side.

33. A method for placing at least one tube in a tube transport rack defining receiving zones for tubes that open upward, the rack comprising a plurality of carriers as claimed in claim 30, in which said tube is inserted in a receiving zone in such a way that the corresponding insert can act on the tube and prevent the rotation of said tube about the longitudinal axis thereof in one or both directions of rotation.
